SEQUENCE LISTING (7 sequences)

SEQ ID NO: 1 - DNA, 18 bp, synthetic construct
  source 1..18: mol_type = other DNA
agtggaggtgaaaattcc

SEQ ID NO: 2 - DNA, 20 bp, synthetic construct
  source 1..20: mol_type = other DNA
ataagagccaaagcccttac

SEQ ID NO: 3 - DNA, 20 bp, synthetic construct
  source 1..20: mol_type = other DNA
tgcatgaatggcgtaacgag

SEQ ID NO: 4 - DNA, 21 bp, synthetic construct
  source 1..21: mol_type = other DNA
gcccgtgcataggcgtatttt

SEQ ID NO: 5 - DNA, 21 bp, synthetic construct
  source 1..21: mol_type = other DNA
attctggcttcagtgtaacgc

SEQ ID NO: 6 - DNA, 21 bp, synthetic construct
  source 1..21: mol_type = other DNA
tttrgcttattcmatgagcgt

SEQ ID NO: 7 - DNA, 20 bp, synthetic construct
  source 1..20: mol_type = other DNA
gcagacggcttggtaraata

(19) United States
(12) Patent Application Publication
(10) Pub. No.: US 2025/0327137 A1
(43) Pub. Date: Oct. 23, 2025

(54) REAGENTS AND METHODS FOR PRESERVATION AND DETECTION OF GASTRIC HELICOBACTER PYLORI

(71) Applicants: National Taiwan University Hospital, Taipei (TW); NATIONAL TAIWAN UNIVERSITY, Taipei (TW)

(72) Inventors: JYH-MING LIOU, Taipei (TW); Ming-Shiang WU, Taipei (TW); Mei-Jyh CHEN, Taipei (TW); Chieh-Chang Chen, Taipei (TW)

(21) Appl. No.: 18/641,601

(22) Filed: Apr. 22, 2024

(51) Int. Cl. C12Q 1/689; C12N 1/04

(52) U.S. Cl. CPC C12Q 1/689; C12N 1/04; C12Q 2600/106; C12Q 2600/156

(57) ABSTRACT

The present disclosure provides methods for effectively preserving test samples utilized in detecting Helicobacter pylori. The present disclosure also provides methods for accurately detecting antibiotic-resistant genotypes of Helicobacter pylori, as well as methods for treating Helicobacter pylori infected-diseases. The disclosed methods effectively improve the efficiency and practicality of both detecting and treating antibiotic-resistant Helicobacter pylori.

### FIELD

The present disclosure generally relates to methods for detecting and treating antibiotic-resistant genotypes of Helicobacter pylori, and more particularly to methods for preserving test samples, as well as primer sets, reagent kits, and methods for detecting antibiotic-resistant genotypes of Helicobacter pylori, and guided methods for treating Helicobacter pylori.

### BACKGROUND

Helicobacter pylori infection is an important pathogenic factor for gastric cancer and peptic ulcer disease, and is associated with immune thrombocytopeniarpura and functional dyspepsia. Eradication of Helicobacter pylori may reduce the risk of gastric cancer, decrease the recurrence rate of peptic ulcer disease, and alleviate symptoms of functional dyspepsia.

With the increasing antibiotic resistance of Helicobacter pylori worldwide, the efficacy of empirical therapy for Helicobacter pylori eradication has gradually decreased. Although culture-based antimicrobial susceptibility testing-guided therapy was shown to be superior to empirical therapy in first-line treatment of Helicobacter pylori infection, culture-based antimicrobial susceptibility testing is time-consuming and inconvenient, limiting its availability in clinical applications.

Studies have shown that point mutations in the 23S ribosomal RNA (23S rRNA) of Helicobacter pylori are associated with clarithromycin resistance, while point mutations in gyrA are associated with levofloxacin resistance. Some studies have used molecular testing for these two gene mutations as the basis for guiding treatment. However, the current eradication rate of molecular testing-guided therapy is only 78%, only slightly higher than the 72% eradication rate of empirical therapy, indicating that there is much room for improvement in molecular testing-guided therapy for Helicobacter pylori.

### SUMMARY

In view of this, the present disclosure provides methods for effectively preserving test samples utilized in detecting Helicobacter pylori. The present disclosure also provides primer sets, reagent kits, and methods for successfully and accurately detecting antibiotic-resistant genotypes of Helicobacter pylori, as well as methods for treating Helicobacter pylori infected-diseases. The disclosed methods effectively improve the efficiency and practicality of both detecting and treating antibiotic-resistant Helicobacter pylori.

In the first part of the present disclosure, a method for preserving a test sample for detecting Helicobacter pylori is provided. The method includes preserving the test sample in a brucella broth.

In some implementation of the present disclosure, the test sample may be a gastric biopsy sample, a gastric fluid sample, or a fecal sample, and a concentration of the brucella broth may be 28 g/L.

In the second part of the present disclosure, a primer set for detecting antibiotic-resistant genotypes of Helicobacter pylori is provided. The primer set includes a first primer set targeting 23S rRNA, a second primer set targeting gyrA, or a combination thereof; wherein the first primer set may include primers with sequences set forth in SEQ ID NO. 1 and SEQ ID NO. 2 or sequences with greater than 90% identity thereto; wherein the second primer may include primers with sequences set forth in SEQ ID NO. 4 and SEQ ID NO. 5 or sequences with greater than 90% identity thereto.

In the third part of the present disclosure, a reagent kit for detecting antibiotic-resistant genotypes of Helicobacter pylori is provided. The reagent kit includes the primer set as described in the second part.

In the fourth part of the present disclosure, a method for detecting antibiotic-resistant genotypes of Helicobacter pylori is provided. The method includes: (a) providing a test sample; (b) providing a reagent kit, which includes the primer set as described in the second part; (c) performing a polymerase chain reaction on the test sample using the primer set of the reagent kit to obtain a product; and (d) analyzing whether the product contains a 23S rRNA mutation, a gyrA mutation, or a combination of both mutations of Helicobacter pylori.

In some implementation of the present disclosure, the test sample may be a gastric biopsy sample, a gastric fluid sample, or a fecal sample preserved by the method as described in the first part.

In some implementation of the present disclosure, the polymerase chain reaction may be set to: react at 95° C. for 1 minute, then react at 95° C. for 30 seconds, 65° C. for 30 seconds, and 68° C. for 30 seconds for 10 cycles, then react at 95° C. for 30 seconds, 59° C. for 30 seconds, and 68° C. for 30 seconds for 38 cycles, and finally react at 68° C. for 10 minutes and then set to store at 4° C.

In some implementation of the present disclosure, the polymerase chain reaction may be set to: react at 95° C. for 1 minute, then react at 95° C. for 30 seconds, 59° C. for 30 seconds, and 68° C. for 30 seconds for 10 cycles, then react at 95° C. for 30 seconds, 55° C. for 30 seconds, and 68° C. for 30 seconds for 38 cycles, and finally react at 68° C. for 10 minutes and then set to store at 4° C.

In the fifth part of the present disclosure, a method for sequentially detecting antibiotic-resistant genotypes of Helicobacter pylori is provided. The method includes: (a) providing a test sample; (b) performing the method as described in the fourth part on the test sample to confirm whether it contains a 23S rRNA mutation of Helicobacter pylori; (b) when no 23S rRNA mutation is detected, determining that the test sample does not contain clarithromycin-resistant Helicobacter pylori, and when a 23S rRNA mutation is detected, determining that the test sample contains clarithromycin-resistant Helicobacter pylori; (c) when the test sample is determined to contain the 23S rRNA mutation, further performing the detection method as described in the fourth part on the test sample to confirm whether it contains a gyrA mutation of Helicobacter pylori; and (d) when no gyrA mutation is detected, determining that the test sample does not contain levofloxacin-resistant Helicobacter pylori, and when a gyrA mutation is detected, determining that the test sample contains levofloxacin-resistant Helicobacter pylori.

In the sixth part of the present disclosure, a method for sequentially detecting antibiotic-resistant genotypes of Helicobacter pylori is provided. The method includes: (a) providing a test sample; (b) performing the detection method as described in the fourth part on the test sample to confirm whether it contains a gyrA mutation of Helicobacter pylori;

(b) when no gyrA mutation is detected, determining that the test sample does not contain levofloxacin-resistant Helicobacter pylori, and when a gyrA mutation is detected, determining that the test sample contains levofloxacin-resistant Helicobacter pylori; (c) when the test sample is determined to contain the gyrA mutation, further performing the detection method as described in the fourth part on the test sample to confirm whether it contains a 23S rRNA mutation of Helicobacter pylori; and (d) when no 23S rRNA mutation is detected, determining that the test sample does not contain clarithromycin-resistant Helicobacter pylori, and when a 23S rRNA mutation is detected, determining that the test sample contains clarithromycin-resistant Helicobacter pylori.

In the seventh part of the present disclosure, a method for treating Helicobacter pylori infected diseases is provided. The method includes: (a) utilizing the method as described in the fourth part to detect whether a subject infected with Helicobacter pylori contains a 23S rRNA mutation; (b) when no 23S rRNA mutation is detected, administering clarithromycin sequential therapy to the subject, and when a 23S rRNA mutation is detected, utilizing the method as described in the fourth part to detect whether the subject infected with Helicobacter pylori contains a gyrA mutation; and (c) when no gyrA mutation is detected, administering levofloxacin sequential therapy to the subject, and when a gyrA mutation is detected, administering bismuth quadruple therapy to the subjects.

In the eighth part of the present disclosure, a method for treating Helicobacter pylori infected diseases is provided. The method includes: (a) utilizing the method as described in the fourth part to detect whether subject infected with Helicobacter pylori contains a gyrA mutation; (b) when no gyrA mutation is detected, administering levofloxacin sequential therapy to the subject, and when a gyrA mutation is detected, utilizing the method as described in the fourth part to detect whether the subject infected with Helicobacter pylori contains a 23S rRNA mutation; and (c) when no 23S rRNA mutation is detected, administering clarithromycin sequential therapy to the subject, and when a 23S rRNA mutation is detected, administering bismuth quadruple therapy to the subject.

In some implementation of the present disclosure, the 23S rRNA mutation may include a mutation at base pair positions 2142 or 2143, such as point mutations of A2143G, A2142G, or A2142C; and the gyrA mutation may include at least one mutation at amino acid positions 87, 88, 91, and 97.

### BRIEF DESCRIPTION OF THE DRAWINGS

The present description will be better understood from the following detailed description when read in conjunction with the appended drawings.

FIG. 1 is a schematic diagram of a first-line treatment process for Helicobacter pylori according to an implementation of the present disclosure.

FIG. 2 is a schematic diagram of a third-line treatment process for Helicobacter pylori according to an implementation of the present disclosure.

### DETAILED DESCRIPTION

The following description contains specific information pertaining to the implementations in the present disclosure. The drawings in the present disclosure and their accompanying detailed description are directed to merely exemplary implementations. However, the present disclosure is not limited to these exemplary implementations. Those skilled in the art will recognize other variations and implementations.

The terms “at least one implementation”, “an implementation”, “some implementations”, “different implementations”, “this implementation”, etc., may indicate that the implementation(s) of the present disclosure so described may include a particular feature, constituent, or characteristic, but not every implementation necessarily includes the particular feature, constituent, or characteristic. Furthermore, repeated use of the phrase “in one implementation” or “in this implementation” does not necessarily refer to the same implementation, although it may. In addition, when phrases such as “implementation” are used in association with “present disclosure”, it does not imply that all implementations of the present disclosure must include the specific features, constituents, or characteristics, but should be understood as “at least some implementations” of the present disclosure include the stated specific features, constituents, or characteristics.

The terms “first”, “second”, “third”, etc. are used to distinguish between different objects, and are not used to describe a particular order. When the term “includes” or “contains” is used, it means “includes but is not limited to”, which explicitly indicates the open relationship between the combination, group, series and equivalents.

First-Line, Second-Line, and Third-Line Treatments of Helicobacter pylori

In the present disclosure, the first-line treatment refers to the initial therapeutic regimen administered to a patient, while the second-line treatment refers to the subsequent therapeutic regimen administered to the patient when the first-line treatment fails (e.g., due to ineffectiveness from the outset or loss of efficacy over time). Similarly, when the second-line treatment also fails, the patient needs to be administered with the third therapeutic regimen, which is referred to as the third-line treatment in the present disclosure. In most cases, antibiotics commonly used in the first-line treatment of Helicobacter pylori include amoxicillin, clarithromycin, and metronidazole; the second-line and third-line treatments often involve bismuth-containing quadruple therapy (e.g., containing tetracycline) or levofloxacin-containing triple therapy.

The first-line treatment of Helicobacter pylori typically involves the use of clarithromycin-based therapy or bismuth quadruple therapy. As for the second-line treatment, levofloxacin-based therapy or bismuth quadruple therapy is commonly recommended. In the present disclosure, sequential therapy with complex prescriptions is employed following antimicrobial susceptibility testing or molecular testing to effectively eradicate Helicobacter pylori strains resistant to clarithromycin or levofloxacin. In cases where antimicrobial susceptibility testing or molecular testing is not performed, bismuth quadruple therapy is the recommended course of action.

### Intention-to-Treat Analysis

In the present disclosure, the intention-to-treat analysis refers to the randomization of subjects and the inclusion of all subjects in the final analysis based on the initially assigned treatment plan rather than the treatment eventually received. This approach aims to avoid the non-random reduction of subjects that may occur in intervention studies (e.g., per-protocol analysis).

### Per-Protocol Analysis

In the present disclosure, the per-protocol analysis refers to the randomization of subjects but the exclusion of subjects who do not comply with the protocol for analysis. This includes subjects who fail to meet the inclusion criteria, analysis criteria, or do not complete the treatment. This approach aims to minimize data processing issues arising from subject loss and to avoid certain estimations, interpolations, and assumptions associated with the intention-to-treat analysis.

### Trial Design and Subject Recruitment

In the present disclosure, two sets of trials were conducted for the first-line and the third-line treatments of Helicobacter pylori, respectively, to evaluate the efficacy of the Helicobacter pylori treatment methods disclosed herein. Both Trial 1 and Trial 2 were multicenter, two-arm, parallel assignment, open-label, randomized controlled trials. The proposals for the trials have been approved by the Review Board of each participating medical institution. Written informed consent has been obtained from all participants before recruitment.

Trial 1 was conducted at seven hospitals and included treatment-naïve subjects aged 20 years or older who were infected with Helicobacter pylori and met the following criteria. Trial 2 was conducted at six hospitals and included subjects aged 20 years or older who met the following criteria and had failed treatment after two or more attempts to eradicate Helicobacter pylori infection. Subjects were excluded from Trial 1 and Trial 2 if they were younger than 20 years old, had a history of gastrectomy or gastric malignancy (including adenocarcinoma or lymphoma), had a previous allergic reaction or contraindication to antibiotics (e.g., amoxicillin, clarithromycin, levofloxacin, and metronidazole) or bismuth or proton pump inhibitors (e.g., esomeprazole), were pregnant or breastfeeding, or had severe concurrent diseases (e.g., end-stage renal failure or decompensated liver cirrhosis). All subjects participating in the trials were diagnosed with Helicobacter pylori infection at outpatient clinics of each study site and were recruited into the trials of the present disclosure.

In Trial 1 and Trial 2, subjects who met the aforementioned criteria were randomly assigned (1:1) to receive either molecular testing-guided therapy (MTGT) or susceptibility (or minimum inhibitory concentration) testing-guided therapy (STGT). The subjects received eradication therapy after the results of molecular testing or susceptibility testing were available. For example, based on the available results of molecular testing or traditional susceptibility testing for resistance to clarithromycin and levofloxacin, the subjects received one or more of clarithromycin sequential therapy, levofloxacin sequential therapy, or bismuth quadruple therapy.

### 1. Trial Procedures

In the present disclosure, a subject infected with Helicobacter pylori was defined as a subject who had two positive results out of the ¹³C-urease breath test (¹³C-UBT), rapid urease test, histology, or culture before being enrolled in the trial. In the 13C-urease breath test used to detect Helicobacter pylori infection, a change value ≥4 units was considered positive, while <2.5 units was considered negative. For subjects with indeterminate test results, another 13C-urease breath test was performed at least 2 weeks later until the results become determinate. All subjects were instructed not to take antibiotics for 4 weeks and not to use proton pump inhibitors for 2 weeks before these tests. At the same time, 13C-UBT was performed again at least 6 weeks after the eradication therapy in accordance with the treatment methods of the present disclosure to determine the eradication rate of Helicobacter pylori.

1.1 Trial 1 (First-Line Treatment of Helicobacter pylori)

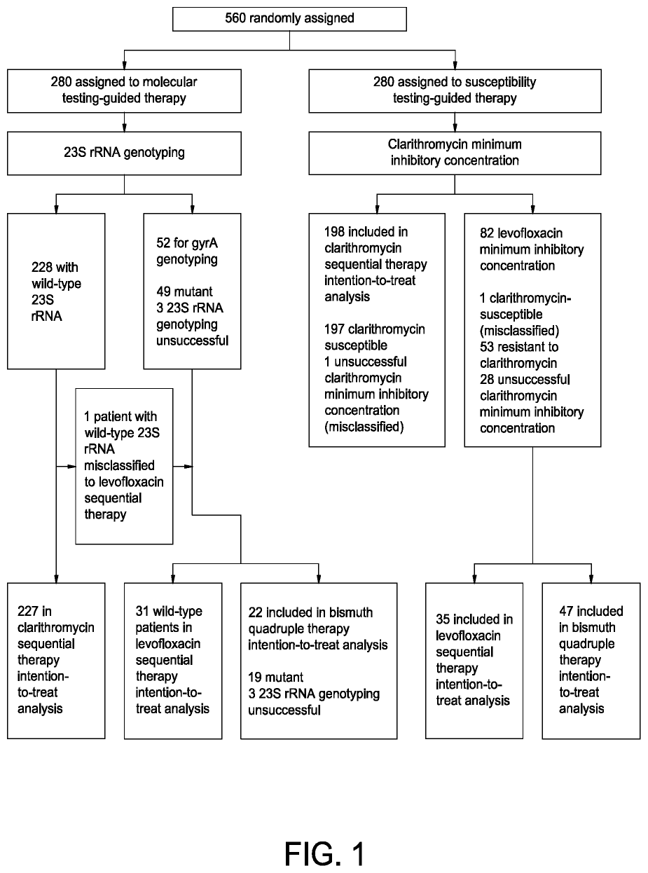

In Trial 1 of the present disclosure, as shown in FIG. 1, a total of 560 treatment-naïve subjects with Helicobacter pylori infection who met the above criteria were recruited and randomly assigned in a 1:1 ratio (i.e., each group included 280 subjects) to the molecular testing-guided therapy group or the susceptibility testing-guided therapy group. In Trial 1, 272 men and 288 women were recruited, with a mean age of 50.9 years (standard deviation 12.9) in the molecular testing-guided therapy group and 53.4 years (standard deviation 13.6) in the susceptibility testing-guided therapy group.

In the molecular testing-guided therapy group of Trial 1, Helicobacter pylori 23S rRNA mutation gene testing was performed first, followed by Gyrase A (hereinafter referred to as gyrA) mutation gene testing to determine the subsequent treatment regimen. More specifically, if the Helicobacter pylori infecting the subject did not have a 23S rRNA mutation gene, clarithromycin sequential therapy was recommended. In cases where a 23S rRNA mutation gene was present, further testing for the gyrA mutation gene was performed. If the Helicobacter pylori infecting the subject did not have a gyrA mutation gene, levofloxacin sequential therapy was recommended. In cases where a gyrA mutation gene was present, bismuth quadruple therapy was recommended.

In the susceptibility testing-guided therapy group of Trial 1, clarithromycin resistance testing was performed first, followed by levofloxacin resistance testing to determine the subsequent treatment regimen. More specifically, if the Helicobacter pylori infecting the subject was not resistant to clarithromycin, clarithromycin sequential therapy was recommended. In cases where clarithromycin resistance was present, further testing for levofloxacin resistance was performed. If the Helicobacter pylori infecting the subject was not resistant to levofloxacin, levofloxacin sequential therapy was recommended. In cases where levofloxacin resistance was present, bismuth quadruple therapy was recommended.

In some implementations, gastric biopsy samples (e.g., gastric biopsy specimens) were used for molecular testing and susceptibility testing of Helicobacter pylori, where the gastric biopsy samples may be obtained by endoscopic biopsy of the gastric mucosa. In other alternative implementations, gastric fluid samples or fecal samples were used for molecular testing and susceptibility testing of Helicobacter pylori. In some implementations, 5 to 7 gastric biopsy samples were obtained for each subject, including 2 samples from the pylorus, used for molecular testing and susceptibility testing after culture, respectively; 2 to 4 samples from the pylorus and gastric corpus, used for histological examination by hematoxylin and eosin (H&E) staining; and 1 sample from the pylorus or gastric corpus, used for rapid urease testing.

In some implementations, molecular testing may involve the use of a DNA purification kit to extract Helicobacter pylori DNA from gastric biopsy samples. Subsequently, amplification of the 23S rRNA fragment and gyrA fragment was carried out using polymerase chain reaction (PCR). Direct sequencing using an automated sequencer was then employed to determine the mutation genes of 23S rRNA and gyrA in Helicobacter pylori. Point mutations observed in the 23S rRNA of Helicobacter pylori were categorized as 23S rRNA mutations, especially mutations at base pair positions 2142 or 2143, such as A2143G, A2142G, and A2142C, indicating Helicobacter pylori resistant to clarithromycin; while mutations affecting amino acid positions 87, 88, 91, and 97 in the gyrA of Helicobacter pylori were categorized as gyrA mutations, indicating Helicobacter pylori resistant to levofloxacin.

In some implementations, susceptibility testing may commence by grinding the gastric biopsy tissue and inoculating it into CDC anaerobic blood agar, which was coated with vancomycin, trimethoprim, and polymyxin B. The inoculated agar was then placed in a microaerophilic incubator for 3 to 4 days. If colonies were produced, 2 to 3 single colony selections were performed, and the presence of Helicobacter pylori was confirmed by observing whether the urea agar slant turns red. Following confirmation, a sterilized cotton swab was used to collect a small amount of the colony, which was then directly smeared onto fresh sheep blood culture medium (brucella broth) for further mass culture. Typically, one original plate may be divided into 4 to 5 plates for culture. Subsequently, numbering was carried out, and the samples were stored at −80° C. for later use. Following the culture process, the minimum inhibitory concentration (MIC) test was employed to determine the antibiotic resistance of Helicobacter pylori. The specific steps for this procedure were as follows: Firstly, the previously mass-cultured bacterial solution was placed in a microaerophilic incubator for 2 to 3 days to activate the strain. On the day of testing, M-H medium was prepared and sterilized, and 5% sheep blood was added in proportion. Antibiotics were then diluted according to the concentration range of each antibiotic. The bacterial solution was subsequently diluted to achieve a bacterial count of approximately 10⁷ to 10⁸ cells/mL, and it was added to the wells replicated on the M-H medium. The plates were then incubated in a microaerophilic incubator for 72 hours. Finally, the results were observed, with the threshold values for resistance defined as follows: clarithromycin ≥1 mg/L, levofloxacin ≥1 mg/L, amoxicillin ≥0.5 mg/L, metronidazole ≥8 mg/L, tetracycline ≥0.5 mg/L, and rifabutin ≥0.5 mg/L.

As shown in FIG. 1, in the molecular testing-guided therapy group of Trial 1, 227 subjects were guided to receive clarithromycin sequential therapy, 31 subjects receive levofloxacin sequential therapy, and 22 subjects receive bismuth quadruple therapy, based on the results of molecular testing for mutations in the 23S rRNA fragment and gyrA fragment. In contrast, in the susceptibility testing-guided therapy group, 198 subjects were guided to receive clarithromycin sequential therapy, 35 subjects receive levofloxacin sequential therapy, and 47 subjects receive bismuth quadruple therapy, based on the results of resistance testing for clarithromycin and levofloxacin.

In Trial 1 of the present disclosure, to effectively monitor treatment efficacy, every participating subject in both the molecular testing-guided therapy group and the susceptibility testing-guided therapy group underwent both molecular testing and minimum inhibitory concentration testing. During the course of Trial 1, subjects underwent five visits, with the first visit being screening, occurring within 1 to 7 days after enrollment; the second visit including endoscopic examination and gastric biopsy sampling for subsequent molecular testing and susceptibility testing, with results available within 1 to 5 weeks; the third visit including dispensing of eradication therapy prescription, lasting 10 to 14 days; the fourth visit being a post-treatment interview at the end of eradication therapy; and the fifth visit including a post-eradication 13C-UBT at 6 to 8 weeks after eradication therapy, with results available within 1 week.

1.2 Trial 2 (Third-Line Treatment of Helicobacter pylori)

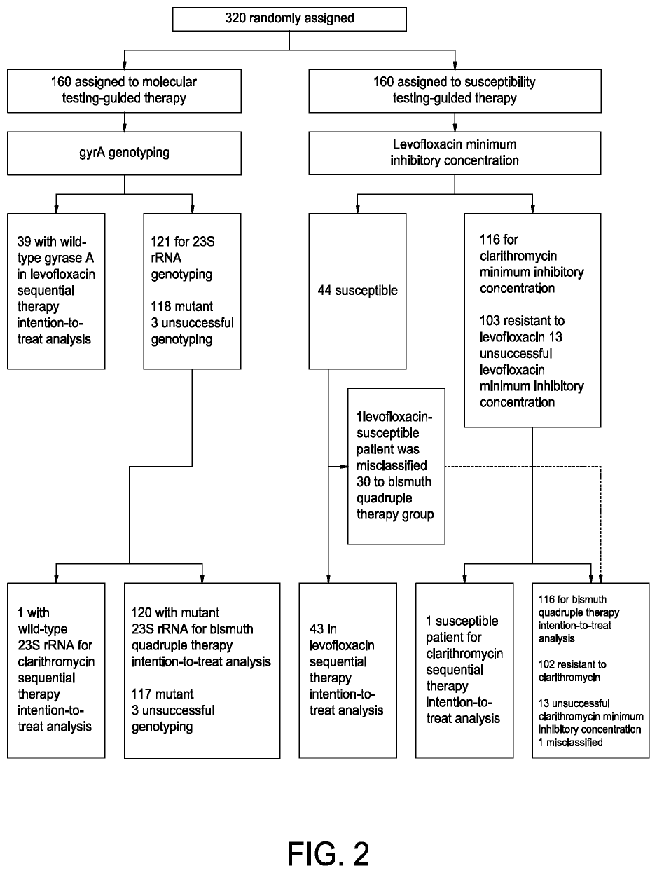

In Trial 2 of the present disclosure, as shown in FIG. 2, a total of 320 subjects with refractory Helicobacter pylori infection who met the aforementioned criteria were recruited and randomly assigned in a 1:1 ratio (i.e., each group included 160 subjects) to the molecular testing-guided therapy group or the susceptibility testing-guided therapy group. In Trial 2, 98 men and 222 women were recruited, with a mean age of 54.1 years (standard deviation 11.4) in the molecular testing-guided therapy group and 53.4 years (standard deviation 10.9) in the susceptibility testing-guided therapy group.

In the molecular testing-guided therapy group of Trial 2, Helicobacter pylori gyrA mutation gene testing was performed first, followed by 23S rRNA mutation gene testing to determine the subsequent treatment regimen. More specifically, if the Helicobacter pylori infecting the subject did not have a gyrA mutation gene, levofloxacin sequential therapy was recommended. In cases where a gyrA mutation gene was present, further testing for the 23S rRNA mutation gene was performed. If the Helicobacter pylori infecting the subject did not have a 23S rRNA mutation gene, clarithromycin sequential therapy was recommended. In cases where a 23S rRNA mutation gene was present, bismuth quadruple therapy was recommended.

In the susceptibility testing-guided therapy group of Trial 2, levofloxacin resistance testing was performed first, followed by clarithromycin resistance testing to determine the subsequent treatment regimen. More specifically, if the Helicobacter pylori infecting the subject was not resistant to levofloxacin, levofloxacin sequential therapy was recommended. In cases where levofloxacin resistance was present, further testing for clarithromycin resistance was performed. If the Helicobacter pylori infecting the subject was not resistant to clarithromycin, clarithromycin sequential therapy was recommended. In cases where clarithromycin resistance was present, bismuth quadruple therapy was recommended.

In some implementations, gastric biopsy samples (e.g., gastric biopsy specimens) were used for molecular testing and susceptibility testing of Helicobacter pylori, where the gastric biopsy samples may be obtained by endoscopic biopsy of the gastric mucosa. In other alternative implementations, gastric fluid samples or fecal samples were used for molecular testing and susceptibility testing of Helicobacter pylori. In some implementations, 5 to 7 gastric biopsy samples were obtained for each subject, including 2 samples from the pylorus, used for molecular testing and susceptibility testing after culture, respectively; 2 to 4 samples from the pylorus and gastric corpus, used for histological examination by H&E staining; and 1 sample from the pylorus or gastric corpus, used for rapid urease testing.

In some implementations, molecular testing may involve the use of a DNA purification kit to extract Helicobacter pylori DNA from gastric biopsy samples. Subsequently, amplification of the 23S rRNA fragment and gyrA fragment was carried out using polymerase chain reaction. Direct sequencing using an automated sequencer was then employed to determine the mutation genes of 23S rRNA and gyrA in Helicobacter pylori. Point mutations observed in the 23S rRNA of Helicobacter pylori were categorized as 23S rRNA mutations, especially mutations at base pair positions 2142 or 2143, such as A2143G, A2142G, and A2142C, indicating Helicobacter pylori resistant to clarithromycin; while mutations affecting amino acid positions 87, 88, 91, and 97 in the gyrA of Helicobacter pylori were categorized as gyrA mutations, indicating Helicobacter pylori resistant to levofloxacin.

In some implementations, susceptibility testing may commence by grinding the gastric biopsy tissue and inoculating it into CDC anaerobic blood agar, which was coated with vancomycin, trimethoprim, and polymyxin B. The inoculated agar was then placed in a microaerophilic incubator for 3 to 4 days. If colonies were produced, 2 to 3 single colony selections were performed, and the presence of Helicobacter pylori was confirmed by observing whether the urea agar slant turns red. Following confirmation, a sterilized cotton swab was used to collect a small amount of the colony, which was then directly smeared onto fresh sheep blood culture medium for further mass culture. Typically, one original plate may be divided into 4 to 5 plates for culture. Subsequently, numbering was carried out, and the samples were stored at −80° C. for later use. Following the culture process, the minimum inhibitory concentration test was employed to determine the antibiotic resistance of Helicobacter pylori. The specific steps for this procedure were as follows: Firstly, the previously mass-cultured bacterial solution was placed in a microaerophilic incubator for 2 to 3 days to activate the strain. On the day of testing, M-H medium was prepared and sterilized, and 5% sheep blood was added in proportion. Antibiotics were then diluted according to the concentration range of each antibiotic. The bacterial solution was subsequently diluted to achieve a bacterial count of approximately 107 to 108 cells/mL, and it was added to the wells replicated on the M-H medium. The plates were then incubated in a microaerophilic incubator for 72 hours. Finally, the results were observed, with the threshold values for resistance defined as follows: clarithromycin ≥1 mg/L, levofloxacin ≥1 mg/L, amoxicillin ≥0.5 mg/L, metronidazole ≥8 mg/L, tetracycline ≥0.5 mg/L, and rifabutin ≥0.5 mg/L.

As shown in FIG. 2, in the molecular testing-guided therapy group of Trial 2, 39 subjects were guided to receive levofloxacin sequential therapy, 1 subject received clarithromycin sequential therapy, and 120 subjects received bismuth quadruple therapy, based on the results of molecular testing for mutations in the 23S rRNA fragment and gyrA fragment. In contrast, in the susceptibility testing-guided therapy group, 43 subjects were guided to receive levofloxacin sequential therapy, 1 subject received clarithromycin sequential therapy, and 116 subjects received bismuth quadruple therapy, based on the results of resistance testing for clarithromycin and levofloxacin.

In Trial 2 of the present disclosure, to effectively monitor treatment efficacy, every participating subject in both the molecular testing-guided therapy group and the susceptibility testing-guided therapy group underwent both molecular testing and minimum inhibitory concentration testing. During the course of Trial 2, subjects underwent five visits, with the first visit being screening, occurring within 1 to 7 days after enrollment; the second visit including endoscopic examination and gastric biopsy sampling for subsequent molecular testing and susceptibility testing, with results available within 1 to 5 weeks; the third visit including dispensing of eradication therapy prescription, lasting 10 to 14 days; the fourth visit being a post-treatment interview at the end of eradication therapy; and the fifth visit including a post-eradication 13C-UBT at 6 to 8 weeks after eradication therapy, with results available within 1 week.

In Trial 2 of the present disclosure, approximately 93% of the subjects had previously received clarithromycin-containing treatment, and 70% of the subjects had previously received levofloxacin-containing treatment. The higher the proportion of subjects who had received clarithromycin- and levofloxacin-containing treatments, the higher the resistance to clarithromycin and levofloxacin. This situation highlights the benefits of employing drug susceptibility testing or molecular testing to plan treatment regimens in order to provide more precise treatment plans at an earlier stage.

### Statistical Analysis of Data

In the present disclosure, eradication rates of the aforementioned Trial 1 and Trial 2 were primarily analyzed and statistically evaluated according to the intention-to-treat analysis. Additionally, the eradication rates were also analyzed and statistically evaluated based on the per-protocol analysis. Simultaneously, the tolerability and frequency of adverse reactions related to the treatment of Helicobacter pylori were also analyzed. Common adverse reactions and medication adherence were assessed at the end of the treatment. Prior to receiving eradication therapy, all subjects were informed about the common adverse reactions associated with the treatment of Helicobacter pylori infection and were requested to record any symptoms during the treatment period to facilitate the evaluation of adverse reactions using a predefined case report form at the end of the treatment.

In the present disclosure, all subjects who were randomly assigned and took the study medications were included in the intention-to-treat analysis. In cases where molecular testing or susceptibility testing failed, bismuth quadruple therapy was administered, and these subjects were included in the intention-to-treat analysis without any further statistical intervention. All subjects who violated the study protocol, such as those who withdrew from the study, took less than 80% of the study medications, were misclassified in the treatment trial, or had an unknown post-treatment Helicobacter pylori status, were excluded from the per-protocol analysis.

In the present disclosure, categorical data were compared using the chi-square test or Fisher's exact test, while continuous data were compared using the student's t-test, and the results were expressed as mean (standard deviation). The p-values were two-tailed, and except for non-inferiority tests, the significance level was defined as p<0.05.

In the present disclosure, according to the non-inferiority trail design, the margin was 5% in Trial 1 and 10% in Trial 2. The eradication rates were evaluated and analyzed using the intention-to-treat analysis and per-protocol analysis. The null hypothesis (H0) of the randomized trial was that the differences in eradication rates were −5% or lower in Trial 1 and −10% or lower in Trial 2. The non-inferiority p-value was calculated in a one-sided test, and if p<0.05, H0 was rejected. In the present disclosure, sensitivity analyses were also performed using the predicted efficacy of the molecular testing-guided therapy group and the susceptibility testing-guided therapy group in regions with different prevalence of clarithromycin and levofloxacin resistance. The eradication rates in regions with different resistance levels were estimated based on the efficacy of resistant and non-resistant strains.

In the present disclosure, statistical analyses were performed using SAS 9.4 for Windows. Trial 1 and Trial 2 were registered on ClinicalTrials.gov with registration numbers NCT03556254 for Trial 1 and NCT03555526 for Trial 2. Although the proportion of clarithromycin resistance differed between the study groups, there was no difference in clarithromycin resistance between the molecular testing-guided therapy group and the susceptibility testing-guided therapy group in Trial 1 (p=0.148) and Trial 2 (p=0.792). The methods for preserving test samples used for detecting Helicobacter pylori, the primer sets and reagent kits for detecting antibiotic-resistant genotypes of Helicobacter pylori, the methods for detecting antibiotic-resistant genotypes of Helicobacter pylori, and the methods for treating Helicobacter pylori infection based on the aforementioned genotypic testing results will be further described through exemplary implementations (i.e., Examples 1 to 4) in the present disclosure.

### Example 1: Preservation of Gastric Biopsy Samples

To optimize the usability of gastric biopsy samples for molecular testing, this exemplary implementation of the present disclosure evaluated various preservation methods, including the absence of preservation solution, and the use of normal saline or brucella broth. It was determined that brucella broth provided the most effective preservation, ensuring optimal detection of Helicobacter pylori. The brucella broth was prepared following the manufacturer's instructions (BD Brucella Broth, product number REF: 211088). In this implementation, 26.6 to 29.4 grams of the broth powder were dissolved in 1 liter of ddH₂O, with a preferred concentration of 28 grams per liter (i.e., 28 g/L). The solution was then dispensed into 2 mL aliquots in 4 mL culture bottles and sterilized using high-temperature and high-pressure methods before use.

Gastric biopsy samples may be obtained by endoscopic biopsy of the gastric mucosa. The obtained gastric biopsy samples, approximately the size of a grain of rice, were then directly stored at 4° C. or immersed in about 2 mL of normal saline or brucella broth and stored at 4° C., with two samples taken for each group. The results showed that gastric biopsy samples preserved in brucella broth could most effectively preserve the samples, and samples preserved in this manner could be directly used for antibiotic resistance gene detection of Helicobacter pylori without the need for further bacterial culture.

In some implementations, when using gastric fluid samples or fecal samples for molecular testing of Helicobacter pylori, the gastric fluid samples or fecal samples may also be collected and preserved using the aforementioned brucella broth as a preservation solution and similarly stored at 4° C.

### Example 2: Detection of 23S rRNA Mutations in Helicobacter pylori

In this exemplary implementation of the present disclosure, the detection of 23S rRNA mutations in Helicobacter pylori was carried out directly on gastric biopsy samples without the need for culturing. The specific method involved the following steps. First, Helicobacter pylori DNA was extracted from gastric biopsy samples using a DNA extraction kit (Gentra Puregene Blood Kit (1000), NO. 158389, QIAGEN, USA), with preference given to samples preserved in brucella broth. The specific extraction procedure was as follows: gastric biopsy tissue was added with 550 μL cell lysis solution and 3.5 μL proteinase K solution, mixed by vortexing, and incubated overnight at 55° C. Subsequently, 4.5 μL RNase A solution was added, mixed by vortexing, and incubated at 35 to 40° C. for 12 to 15 minutes, preferably at 37° C. for 15 minutes. Following this, 200 μL protein precipitation solution was added, mixed by vortexing, and incubated at 4° C. for 25 to 35 minutes, preferably for 30 minutes, followed by centrifugation under appropriate conditions at 4° C. to precipitate the protein. The supernatant was then transferred to a new 1.5 mL centrifuge tube, and an equal volume of isopropanol was added (with an additional 1 μL of glycogen when extracting a small amount of DNA), followed by inversion to allow nucleic acid aggregation. This was followed by centrifugation at 12000 to 14000 rpm for 20 minutes at room temperature, preferably at 13000 rpm. After the supernatant was removed, 900 μL of 70% ethanol was added, and the mixture was centrifuged again at 12000 to 14000 rpm for 20 minutes, preferably at 13000 rpm. The 70% ethanol was then removed, and the samples were air-dried at room temperature. Finally, 100 μL of DNA hydration solution was added, and the mixture was stored at −20° C. for subsequent use. In some implementations, gastric biopsy samples, gastric fluid samples, or fecal samples preserved in brucella broth as described in Example 1 may be used, and DNA extraction may be performed directly using the DNA extraction kit without removing the preservation solution.

Following the extraction of Helicobacter pylori DNA, PCR was conducted to amplify the peptidyltransferase (Hp23S fragment) of 23S rRNA using the forward primer CRF-4 (SEQ ID NO. 1) and the reverse primer CRR-1 (SEQ ID NO. 2) from Table 1. This process aimed to obtain a PCR product of 135 bp. The PCR reaction conditions were set as follows. Initial denaturation at 95° C. for 1 minute, followed by 10 cycles of denaturation at 95° C. for 30 seconds, annealing at 65° C. for 30 seconds, and extension at 68° C. for 30 seconds. Subsequently, 38 cycles of denaturation at 95° C. for 30 seconds, annealing at 59° C. for 30 seconds, and extension at 68° C. for 30 seconds were performed. Finally, the reaction was concluded with an extension step at 68° C. for 10 minutes, followed by storage at 4° C. The Hotstart Taq Premix (w/o dye) (BN0100-0001, Wanwang Biotech) detection kit was utilized for the PCR reactions, and the primers were synthesized by Booster Biotech. The obtained PCR products were outsourced for sequencing using the Applied Biosystems HT ExoSAP-IT High-Throughput PCR Product Cleanup 2743446. The results indicated that employing these PCR reaction conditions for molecular testing to identify clarithromycin-resistant strains enhanced accuracy to 92%.

TABLE 1

| Primer | | |
|---|---|---|
| Name | Nucleotide Sequence | Sequence Number |
| CRF-4 | agtggaggtgaaaattcc | SEQ ID NO. 1 |
| CRR-1 | ataagagccaaagcccttac | SEQ ID NO. 2 |
| Hp23F2 | tgcatgaatggcgtaacgag | SEQ ID NO. 3 |

In this implementation of the present disclosure, PCR was conducted using the forward primer Hp23F2 (SEQ ID NO. 3) and the reverse primer CRR-1 (SEQ ID NO. 2) from Table 1 to amplify the peptidyltransferase of 23S rRNA in the extracted Helicobacter pylori DNA, resulting in a PCR product of 200 bp for comparison. The PCR reaction conditions were set as follows. Initial denaturation at 95° C. for 10 minutes, followed by 35 cycles of denaturation at 95° C. for 30 seconds, annealing at 50˜60° C. for 30 seconds, and extension at 72° C. for 45 seconds. Subsequently, a final extension step at 72° C. for 10 minutes was performed, followed by storage at 4° C. The preferred annealing temperature for the PCR reaction was 52° C. The PCR product was purified using a reagent kit and subjected to direct sequencing using an automated sequencer. The results indicated that employing these PCR reaction conditions for molecular testing to identify clarithromycin-resistant strains yielded an accuracy of only 85%.

### Example 3: Detection of gyrA Mutations in Helicobacter pylori

In this exemplary implementation of the disclosure, the detection of gyrA mutations in Helicobacter pylori was carried out directly on gastric biopsy samples without the need for culturing. The specific method involved the following steps. First, Helicobacter pylori DNA was extracted from gastric biopsy samples using a DNA extraction kit (Gentra Puregene Blood Kit (1000), NO. 158389, QIAGEN, USA), with preference given to samples preserved in brucella broth. The specific extraction procedure was as follows: gastric biopsy tissue was added with 550 μL cell lysis solution and 3.5 μL proteinase K solution, mixed by vortexing, and incubated overnight at 55° C. Subsequently, 4.5 μL RNase A solution was added, mixed by vortexing, and incubated at 35 to 40° C. for 12 to 15 minutes, preferably at 37° C. for 15 minutes. Following this, 200 μL protein precipitation solution was added, mixed by vortexing, and incubated at 4° C. for 25 to 35 minutes, preferably for 30 minutes, followed by centrifugation under appropriate conditions at 4° C. to precipitate the protein. The supernatant was then transferred to a new 1.5 mL centrifuge tube, and an equal volume of isopropanol was added (with an additional 1 μL of glycogen when extracting a small amount of DNA), followed by inversion to allow nucleic acid aggregation. This was followed by centrifugation at 12000 to 14000 rpm for 20 minutes at room temperature, preferably at 13000 rpm. After the supernatant was removed, 900 μL of 70% ethanol was added, and the mixture was centrifuged again at 12000 to 14000 rpm for 20 minutes, preferably at 13000 rpm. The 70% ethanol was then removed, and the samples were air-dried at room temperature. Finally, 100 μL of DNA hydration solution was added, and the mixture was stored at-20° C. for subsequent use. In some implementations, gastric biopsy samples, gastric fluid samples, or fecal samples preserved in brucella broth as described in Example 1 may be used, and DNA extraction may be performed directly using the DNA extraction kit without removing the preservation solution.

Following the extraction of Helicobacter pylori DNA, PCR was conducted to amplify the gyrA fragment using the forward primer gyrAF5 (SEQ ID NO. 4) and the reverse primer gyrAR5 (SEQ ID NO. 5) from Table 2. This process aimed to obtain a PCR product of 255 bp. The PCR reaction conditions were set as follows. Initial denaturation at 95° C. for 1 minute, followed by 10 cycles of denaturation at 95° C. for 30 seconds, annealing at 59° C. for 30 seconds, and extension at 68° C. for 30 seconds. Subsequently, 38 cycles of denaturation at 95° C. for 30 seconds, annealing at 55° C. for 30 seconds, and extension at 68° C. for 30 seconds were performed. Finally, the reaction was concluded with an extension step at 68° C. for 10 minutes, followed by storage at 4° C. The Hotstart Taq Premix (w/o dye) (BN0100-0001, Wanwang Biotech) detection kit was utilized for the PCR reactions, and the primers were synthesized by Booster Biotech. The obtained PCR products were outsourced for sequencing using the Applied Biosystems HT ExoSAP-IT High-Throughput PCR Product Cleanup 2743446. The results indicated that employing these PCR reaction conditions for molecular testing to identify levofloxacin-resistant strains enhanced accuracy to 95%.

TABLE 2

| Primer | | |
|---|---|---|
| Name | Nucleotide Sequence | Sequence Number |
| gyrAF5 | gcccgtgcataggcgtatttt | SEQ ID NO. 4 |
| gyrAR5 | attctggcttcagtgtaacgc | SEQ ID NO. 5 |
| gyrA1F | tttrgcttattcmatgagcgt | SEQ ID NO. 6 |
| gyrA1R | gcagacggcttggtaraata | SEQ ID NO. 7 |

In this implementation of the present disclosure, PCR was conducted using the forward primer gyrA1F (SEQ ID NO. 6) and the reverse primer gyrA1R (SEQ ID NO. 7) from Table 2 to amplify the gyrA fragment in the extracted Helicobacter pylori DNA, resulting in a PCR product of 428 bp for comparison. The PCR reaction conditions were set as follows. Initial denaturation at 95° C. for 10 minutes, followed by 35 cycles of denaturation at 95° C. for 30 seconds, annealing at 50˜60° C. for 30 seconds, and extension at 72° C. for 45 seconds. Subsequently, a final extension step at 72° C. for 10 minutes was performed, followed by storage at 4° C. The preferred annealing temperature for the PCR reaction was 52° C. The PCR product was purified using a reagent kit and subjected to direct sequencing using an automated sequencer. The results indicated that employing these PCR reaction conditions for molecular testing to identify levofloxacin-resistant strains yielded an accuracy of only 85-90%.

### Example 4: Treatment of Helicobacter pylori Infection

In this exemplary implementation of the present disclosure, the clarithromycin sequential therapy for subjects in Trial 1 and Trial 2 may be a 14-day clarithromycin sequential therapy: esomeprazole 40 mg and amoxicillin 1 g were initially taken twice daily for 7 days, followed by esomeprazole 40 mg, clarithromycin 500 mg, and metronidazole 500 mg, taken twice daily for another 7 days. The levofloxacin sequential therapy may be a 14-day levofloxacin sequential therapy: esomeprazole 40 mg and amoxicillin 1 g were initially taken twice daily for 7 days, followed by esomeprazole 40 mg, levofloxacin 250 mg, and metronidazole 500 mg, taken twice daily for another 7 days. The bismuth quadruple therapy may be a 10-day bismuth quadruple therapy: esomeprazole 40 mg taken twice daily, metronidazole 500 mg taken three times daily, bismuth potassium citrate 300 mg (KCB FC tablets, Swiss Pharmaceutical, Tainan, Taiwan) taken four times daily, and tetracycline 500 mg taken four times daily, for a total of 10 days.

After administering the specified treatment regimens to treat the subjects' Helicobacter pylori infection, the resulting data underwent statistical analysis employing the methods previously outlined. The details of this analysis were elaborated upon below. 4.1 First-line Treatment of Helicobacter pylori Infection (Trial 1)

In Trial 1 of the present disclosure, among the total of 560 subjects, 551 underwent successful molecular testing, indicating a high success rate of approximately 98%. This suggests that the molecular testing method of the present disclosure is operationally straightforward and demonstrates excellent feasibility. In contrast, only 494 out of the 560 subjects successfully underwent the susceptibility testing, resulting in a success rate of approximately 88%. This success rate was notably lower than that of molecular testing. Among the 66 subjects who failed susceptibility testing, 26 (about 39%) showed no colony growth in culture, 34 (about 52%) had culture contamination, and 6 (about 9%) had instrument errors.

In Trial 1 of the present disclosure, according to the results of the intention-to-treat analysis, in the molecular testing-guided therapy group, the first-line treatment of Helicobacter pylori infection successfully eradicated 241 out of 280 subjects (86%, 95% CI 82-90), while in the susceptibility testing-guided therapy group, 243 out of 280 subjects were successfully eradicated (87%, 95% CI 83-91) (p=0.81). Similarly, in Trial 1, according to the results of the per-protocol analysis, in the molecular testing-guided therapy group, 240 out of 265 subjects were successfully eradicated (91%, 87-94), while in the susceptibility testing-guided therapy group, 240 out of 262 subjects were successfully eradicated (92%, 88-95) (p=0.68).

In Trial 1 of the present disclosure, using a non-inferiority margin of 5%, according to the results of the intention-to-treat analysis, the difference in eradication rates between the molecular testing-guided therapy group and the susceptibility testing-guided therapy group was −0.7% (95% CI −6.4 to 5.0; non-inferiority p=0.071), while according to the results of the per-protocol analysis, the difference in eradication rates between the two groups was −1.0% (95% CI −5.9 to 3.8; non-inferiority p=0.059).

In Trial 1 of the present disclosure, there was no significant difference in the frequency of adverse reactions between the molecular testing-guided therapy group and the susceptibility testing-guided therapy group. At the same time, there was no significant difference in eradication rates between males and females (p=0.83). In Trial 1 of the present disclosure, according to the results of the intention-to-treat analysis, there was no significant difference in the eradication rate of clarithromycin sequential therapy between the molecular testing-guided therapy group and the susceptibility testing-guided therapy group (p=0.64); at the same time, there was no significant difference in levofloxacin sequential therapy and bismuth quadruple therapy between the molecular testing-guided therapy group and the susceptibility testing-guided therapy group. In addition, there was no significant difference in eradication rates between the molecular testing-guided therapy group and the susceptibility testing-guided therapy group at different medical institutions.

These results show that the molecular testing method of the present disclosure is straightforward to execute, displaying outstanding feasibility and a notably high success rate. By utilizing the results derived from the molecular testing to guide suitable treatment approaches, comparable eradication rates to those achieved with traditional susceptibility testing-guided therapy may be attained in the population with treatment-naïve Helicobacter pylori infection, greatly improving the problem of lower eradication rates with molecular testing-guided therapy in prior art.

4.2 Third-Line Treatment of Helicobacter pylori Infection (Trial 2)

In Trial 2 of the present disclosure, among the total of 320 subjects, 312 underwent successful molecular testing, indicating a high success rate of approximately 98%. This suggests that the molecular testing method of the present disclosure is operationally straightforward and demonstrates excellent feasibility. In contrast, only 295 out of the 320 subjects successfully underwent the susceptibility testing, resulting in a success rate of approximately 92%, which was significantly lower than the success rate of the molecular testing.

In Trial 2 of the present disclosure, according to the results of the intention-to-treat analysis, in the molecular testing-guided therapy group, the third-line treatment of Helicobacter pylori infection successfully eradicated 141 out of 160 subjects (88%, 95% CI 83-93), while in the susceptibility testing-guided therapy group, 139 out of 160 subjects were successfully eradicated (87%, 95% CI 82-92) (p=0.74). Similarly, in Trial 2, according to the results of the per-protocol analysis, in the molecular testing-guided therapy group, 140 out of 155 subjects were successfully eradicated (90%, 86-95), while in the susceptibility testing-guided therapy group, 139 out of 156 subjects were successfully eradicated (89%, 84-94) (p=0.72).

In Trial 2 of the present disclosure, using a non-inferiority margin of 10%, according to the results of the intention-to-treat analysis, the difference in eradication rates between the molecular testing-guided therapy group and the susceptibility testing-guided therapy group was 1.3% (95% CI −6.0 to 8.5; non-inferiority p=0.0018), while according to the results of the per-protocol analysis, the difference in eradication rates between the two groups was 1.2% (95% CI −5.5 to 8.0; non-inferiority p=0.0012). This shows that in the third-line treatment of Helicobacter pylori, the efficacy of the molecular testing-guided therapy group was not inferior to that of the susceptibility testing-guided therapy group.

In Trial 2 of the present disclosure, there was no significant difference in the frequency of adverse reactions between the molecular testing-guided therapy group and the susceptibility testing-guided therapy group. At the same time, there was no significant difference in eradication rates between males and females (p=0.96). In Trial 2 of the present disclosure, according to the results of the intention-to-treat analysis, there was no significant difference in the eradication rates of levofloxacin sequential therapy and bismuth quadruple therapy between the molecular testing-guided therapy group and the susceptibility testing-guided therapy group. In addition, there was no significant difference in eradication rates between the molecular testing-guided therapy group and the susceptibility testing-guided therapy group at different medical institutions.

These results show that the molecular testing method of the present disclosure is straightforward to execute, displaying outstanding feasibility and a notably high success rate. By utilizing the results derived from the molecular testing to guide suitable treatment approaches, comparable eradication rates to those achieved with traditional susceptibility testing-guided therapy may be attained in the population with refractory Helicobacter pylori infection, greatly improving the problem of lower eradication rates with molecular testing-guided therapy in prior art.

The integrated analysis of Trial 1 and Trial 2 showed that the overall estimate of the risk difference between the molecular testing-guided therapy group and the susceptibility testing-guided therapy group was −0.03% (−4.4 to 4.5). Similarly, the overall estimate of the risk difference according to the intention-to-treat analysis and the per-protocol analysis was −0.3% (−4.2 to 3.7).

Overall, in the molecular testing-guided therapy group, the eradication rates of first-line and third-line treatment of Helicobacter pylori were both over 90%, significantly surpassing the 78% eradication rate reported in previous studies of molecular testing-guided therapy for refractory Helicobacter pylori infection.

Although culture-based susceptibility testing-guided therapy may be used to guide the treatment of Helicobacter pylori infection, it involves culturing the bacteria under specific microaerophilic conditions using specialized media for at least 7 to 10 days or even longer, leading to significant variations in culture success rates across different facilities (typically ranging from about 75% to 90%). Additionally, accurate strain identification necessitates specific training, further prolonging the process. Overall, obtaining susceptibility testing results through culture takes at least 4 weeks. In contrast, the molecular testing-guided therapy described in the present disclosure does not rely on bacterial culture, and in addition to using gastric biopsy samples, non-invasive samples such as gastric (intra) fluid or fecal samples may also be utilized for genetic testing. This approach enables the rapid acquisition of relevant information regarding the antibiotic resistance genotypes of Helicobacter pylori within a short timeframe of 4 to 5 days. Consequently, the overall time required for molecular testing results is approximately 1 week, significantly enhancing the efficiency of detecting and treating antibiotic-resistant Helicobacter pylori infections.

Moreover, the utilization of a specialized sample preservation solution in the present disclosure enables direct antibiotic resistance genotyping of Helicobacter pylori from samples such as gastric biopsy specimens without the need for culturing. Coupled with optimized polymerase chain reaction conditions, the overall success rate of testing may reach 98%. The accuracy of molecular testing for 23S rRNA mutations, indicative of clarithromycin resistance, may achieve 95.5%. Similarly, the accuracy of molecular testing for gyrA mutations, indicating levofloxacin resistance, may reach 92.4%. These advancements address the challenge of lower eradication rates associated with molecular testing-guided therapy in previous approaches. This underscores the effective applicability of the present disclosure for detecting and treating antibiotic-resistant Helicobacter pylori infections.

Based on the above description, it is evident that various techniques may be used to implement the concepts described in this application without departing from the scope of these concepts. Furthermore, although the concepts have been described with specific reference to certain implementations, those skilled in the art will recognize that changes may be made in form and detail without departing from the scope of these concepts. Thus, the described implementations will be considered illustrative rather than restrictive in all aspects. Moreover, it should be understood that this application is not limited to the specific implementations described above, and that many rearrangements, modifications, and substitutions may be made without departing from the scope of the present disclosure.

What is claimed is:

1. A method for preserving a test sample for detection of Helicobacter pylori, comprising preserving the test sample in a brucella broth.

2. The method of claim 1, wherein the test sample is a gastric biopsy sample, a gastric fluid sample, or a fecal sample.

3. The method of claim 1, wherein a concentration of the brucella broth is 28 g/L.

4. A method for detecting antibiotic-resistant genotypes of Helicobacter pylori, comprising: (a) providing a test sample; (b) using a primer set to perform a polymerase chain reaction on the test sample to obtain a product; and (c) analyzing whether the product contains a 23S rRNA mutation of Helicobacter pylori; wherein the polymerase chain reaction is set to: react at 95° C. for 1 minute, then react at 95° C. for 30 seconds, 65° C. for 30 seconds, and 68° C. for 30 seconds for 10 cycles, then react at 95° C. for 30 seconds, 59° C. for 30 seconds, and 68° C. for 30 seconds for 38 cycles, and finally react at 68° C. for 10 minutes and then set to store at 4° C.

5. The method of claim 4, wherein the test sample is a gastric biopsy sample, a gastric fluid sample, or a fecal sample preserved by the method of claim 1.

6. The method of claim 4, wherein the primer set comprises a first primer set targeting 23S rRNA, and the first primer set comprises primers with sequences set forth in SEQ ID NO. 1 and SEQ ID NO. 2 or sequences with greater than 90% identity thereto.

7. The method of claim 4, wherein the 23S rRNA mutation includes a mutation at base pair positions 2142 or 2143.

8. A method for detecting antibiotic-resistant genotypes of Helicobacter pylori, comprising: (a) providing a test sample; (b) using a primer set to perform a polymerase chain reaction on the test sample to obtain a product; and (c) analyzing whether the product contains a gyrA mutation of Helicobacter pylori; wherein the polymerase chain reaction is set to: react at 95° C. for 1 minute, then react at 95° C. for 30 seconds, 59° C. for 30 seconds, and 68° C. for 30 seconds for 10 cycles, then react at 95° C. for 30 seconds, 55° C. for 30 seconds, and 68° C. for 30 seconds for 38 cycles, and finally react at 68° C. for 10 minutes and then set to store at 4° C.

9. The method of claim 8, wherein the test sample is a gastric biopsy sample, a gastric fluid sample, or a fecal sample preserved by the method of claim 1.

10. The method of claim 8, wherein the primer set comprises a second primer set targeting gyrA, and the second primer set comprises primers with sequences set forth in SEQ ID NO. 4 and SEQ ID NO. 5 or sequences with greater than 90% identity thereto.

11. The method of claim 8, wherein the gyrA mutation includes at least one mutation at amino acid positions 87, 88, 91, and 97.

12. A method for treating Helicobacter pylori infected diseases, comprising: (a) utilizing the method of claim 4 to detect whether a subject infected with Helicobacter pylori contains a 23S rRNA mutation; (b) when no 23S rRNA mutation is detected, administering clarithromycin sequential therapy to the subject, and when a 23S rRNA mutation is detected, utilizing the method of claim 8 to detect whether the subject infected with Helicobacter pylori contains a gyrA mutation; and (c) when no gyrA mutation is detected, administering levofloxacin sequential therapy to the subject, and when a gyrA mutation is detected, administering bismuth quadruple therapy to the subjects.

13. A method for treating Helicobacter pylori infected diseases, comprising: (a) utilizing the method of claim 8 to detect whether subject infected with Helicobacter pylori contains a gyrA mutation; (b) when no gyrA mutation is detected, administering levofloxacin sequential therapy to the subject, and when a gyrA mutation is detected, utilizing the method of claim 4 to detect whether the subject infected with Helicobacter pylori contains a 23S rRNA mutation; and (c) when no 23S rRNA mutation is detected, administering clarithromycin sequential therapy to the subject, and when a 23S rRNA mutation is detected, administering bismuth quadruple therapy to the subject.
